(19) United States
(12) Patent Application Publication
(10) Pub. No.: US 2026/0275400 A1
(43) Pub. Date: Sep. 17, 2026

(54) METHOD FOR CHARACTERIZING CELLS

(71) Applicant: PATTERN BIOSCIENCE, INC., Austin, TX (US)

(72) Inventors: Natalie A. PACE, Jonestown, TX (US); Bryson L. SEEKINS, Temple, TX (US)

(73) Assignee: PATTERN BIOSCIENCE, INC., Austin, TX (US)

(21) Appl. No.: 19/567,787

(22) Filed: Mar. 16, 2026

Related U.S. Application Data
(60) Provisional application No. 63/772,872, filed on Mar. 17, 2025.

(51) Int. Cl. C12Q 1/14; G01N 21/64

(52) U.S. Cl. CPC C12Q 1/14; G01N 21/6428; G01N 2021/6439

(57) ABSTRACT

The disclosure provides methods for characterizing cells. The methods of the disclosure comprise combining the cells with a growth media to form a sample mixture, partitioning the sample mixture into a plurality of volume partitions, arranging the plurality of volume partitions on at least one array, incubating the volume partitions, monitoring the neighboring volume partitions for one or more characteristics, and thereby characterizing the cells in the sample.

The disclosure relates to methods for characterizing cells.

Characterizing cells is important in clinical diagnosis and treatment. Where a subject is suffering from infection with cells of unknown species, a need exists to accurately characterize said cells to design appropriate treatment. Methods of identifying such cells are essential to allow clinicians to make correct treatment decisions.

Existing methods for characterizing cells require a combination of biochemical tests, mass spectrometry (e.g., matrix-assisted laser desorption ionization time-of-flight mass spectrometry, MALDI-TOF MS), and genetic tests. Exemplary biochemical tests include gram stain, hemolytic activity, catalase activity, bile solubility, and optochin susceptibility. An exemplary genetic test is the rapid genetic test Verigene (Wojewoda et al., Journal of Clinical Microbiology, 2013). Other existing methods are also known in the art.

However, existing methods have several drawbacks in that they typically require multiple days for cell culture, specialized experimental skills, and/or integration of information from multiple systems.

For example, existing methods require monomicrobial samples. Samples must be streaked and cultured for at least 12 hours, typically overnight. Following culture, potential distinct organisms must then be sub-cultured for at least a further 12 hours. Once culture and sub-culture is complete, the existing methods for characterizing cells can be completed.

MALDI-TOF requires about 3 minutes following culture and sub-culture.

Verigene requires about 2-3 hours following culture and sub-culture.

Bile solubility requires about 10 minutes following sub-culture.

Optochin requires a sub-culture of at least about 18 hours following an initial culture of at least about 18 hours.

In addition, existing methods, such as Verigene, cannot be used for characterizing certain cells including S. pneumoniae. Existing methods may only be capable of characterizing certain cells in limited sources, such as mono-microbial sources like blood samples.

Moreover, the accuracy of results from existing methods is sub-optimal.

Therefore, a need exists for improved methods for characterizing cells.

The disclosure is directed to improved methods for characterizing cells. The methods of the disclosure may provide improved sensitivity and/or improved specificity.

### SUMMARY

The disclosure is directed to a method for characterizing cells. In some embodiments, the method comprises (a) combining the cells with a growth media comprising one or more reagents, thereby forming a sample mixture, wherein one or more of the reagents is a detection agent; (b) partitioning the sample mixture into a plurality of volume partitions, some or each of said volume partitions containing one cell or one cellular aggregate from the sample mixture; (c) arranging the plurality of volume partitions on at least one array; (d) incubating the volume partitions for a time sufficient to permit (i) at least one initial reaction to occur to produce at least one product, and (ii) diffusion of one or more of the products into one or more neighboring volume partition(s); (e) monitoring the neighboring volume partitions for one or more characteristics that results from a subsequent reaction between one or more of the products and the detection agent(s); and (f) thereby characterizing the cells in the sample.

In some embodiments of the method, the volume partitions are emulsified droplets.

In some embodiments of the method, incubating the volume partitions comprises incubating at one or more temperatures.

In some embodiments, one or more of the products is a cell metabolite and/or an oxidizing agent.

In some embodiments, the metabolite is hydrogen peroxide and/or the oxidizing agent is oxidized hemoglobin.

In some embodiments, one of the reagents is hemoglobin.

In some embodiments, monitoring the one or more characteristics comprises monitoring an amount of signal produced by a reaction between one or more of the products and the detection agent(s).

In some embodiments, the volume partitions have a volume of less than 1 μL.

In some embodiments, the volume of the partitions is less than 100 nL, 10 nL, 1 nL, 100 p L, 10 pL, 1 pL, 100 fL, 10 fL, or 1 fL.

In some embodiments, one or more of the characteristics are optical characteristics and/or chemical characteristics.

In some embodiments, the optical characteristics are fluorometric, colorimetric and/or reflectance characteristics.

In some embodiments, monitoring the optical characteristics comprises monitoring for a decrease in fluorescent signal in the neighboring volume partitions.

In some embodiments, monitoring one or more of the characteristics comprises constructing a waveform for each volume partition, wherein such a waveform reflects the change of one or more of the characteristics over time.

In some embodiments, the detection agent is a fluorescent dye.

In some embodiments, the fluorescent dye is pyranine.

In some embodiments, the cells are heterogeneously distributed among the volume partitions.

In some embodiments, the cells are bacterial cells.

In some embodiments, the bacterial cells are Streptococcus pneumoniae.

In some embodiments, the growth media is tryptic soy broth.

In some embodiments, the time sufficient to permit the at least one reaction and the diffusion to occur is at least 2 hours, at least 4 hours, or at least 6 hours.

The disclosure is also directed to a method for determining the presence of Streptococcus pneumoniae cells in a sample. In some embodiments, the method comprises (a) combining the sample with a growth media comprising tryptic soy broth, hemoglobin, and a fluorescent dye forming a sample mixture; (b) partitioning the sample mixture into a plurality of membrane emulsified droplets, some or each of said droplets containing one cell or one cellular aggregate from the sample mixture; (c) arranging the plurality of droplets on at least one array; (d) incubating the droplets for a time sufficient to permit (i) metabolism of the Streptococcus pneumoniae cells to produce hydrogen peroxide, (ii) a reaction between the hydrogen peroxide and the hemoglobin to produce oxidized hemoglobin, (iii) a reaction between the oxidized hemoglobin and the fluorescent dye to produce an oxidized dye, and (iv) diffusion of at least one of the hydrogen peroxide, the oxidized hemoglobin, or the oxidized dye into one or more neighboring droplets; (e) monitoring a fluorescent signal from the one or more neighboring droplets during the incubation; and (f) thereby determining the presence or absence of Streptococcus pneumoniae cells in the sample.

In some embodiments of the method for determining the presence of Streptococcus pneumoniae cells in a sample, the fluorescent dye comprises pyranine.

### BRIEF DESCRIPTION OF DRAWING(S)

The drawings are for illustration purposes only and do not provide any limitation on the disclosure.

FIG. 1 illustrates a two-dimensional droplet array. First (1), second (2), and third (3) nearest neighbor droplets for an exemplary droplet (0) are also depicted in FIG. 1.

FIG. 2 illustrates a droplet array in which droplets containing Streptococcus pneumoniae produce a decrease of pyranine fluorescent signal in neighboring droplets.

FIGS. 3A-D illustrate waveforms depicting the differential pyranine signal for droplets comprising Streptococcus pneumoniae cells compared to Streptococcus mitis cells. FIG. 3A depicts the differential signal volume partitions comprising S. pneumoniae cells or S. mitis cells. FIGS. 3B-3D depict the differential signals for the hexagonally close-packed first nearest neighbor droplets (FIG. 3B), second nearest neighbor droplets (FIG. 3C), and third nearest neighbor droplets (FIG. 3D). FIGS. 3A-3D provide differential signal from a representative strain from each species from 14 S. pneumoniae strains and 14 S. mitis strains.

FIG. 4 illustrates kinetic parameter measurements for droplets from a variety of strains, wherein each strain is indicated by a point. The solid horizontal line between the kinetic parameter values 0.7 and 0.8 provides an exemplary means by which an algorithm could distinguish between S. mitis and S. pneumoniae cells to characterize cells.

FIG. 5 illustrates a droplet array for a polymicrobial context wherein the sample comprises Streptococcus pneumoniae and Streptococcus mitis cells. Droplets comprising Streptococcus mitis cells are identified in a separate optical channel are indicated by dashed circles. Droplets comprising Streptococcus pneumoniae are indicated by solid circles.

### DETAILED DESCRIPTION

The disclosure is directed to methods for characterizing cells.

### Definitions

As used herein, “clinically identifying cells” means identifying one or more characteristics of a cell or cells to identify a treatment for a subject infected with said cells.

As used herein, “neighboring volume partitions” refer to volume partitions that are in close proximity to each other. Volume partitions are in close proximity if the volume partitions have a center-to-center distance of approximately the diameter of the volume partition or less. In some embodiments, volume partitions are in close proximity if the volume partitions are within 100 μm of the volume partition from which at least one product diffuses.

As used herein, a “chamber” is a volume partition comprising a semi-permeable membrane that allows diffusion of one or more products.

As used herein, a “colorimetric characteristic” is the amount of light which passes through a sample. In some embodiments, the colorimetric characteristic comprises differential efficiencies of light passing through, differences in the transmission of light, and/or the spectral composition of light.

As used herein, a “detection agent” is any agent capable of being detected.

As used herein, a “fluorescent dye” is any dye capable of producing a fluorescent signal.

As used herein, a “fluorometric characteristic” is a fluorescent signal.

As used herein, “growth media” is any media that allows growth of one or more cells. Growth media comprises all necessary ingredients for the growth of at least one cell type.

As used herein, “hexagonally closely-packed” volume partitions form a hexagonal pattern on the array, wherein each volume partition is surrounded by six volume partitions arranged in a hexagonal ring.

As used herein, “Laplace pressure” is the differential pressure between the inside and outside of a curved surface, such as the difference in pressure between the inside and outside of a droplet.

As used herein, “first nearest neighbor” volume partitions are the volume partitions surrounding the volume partition comprising a cell or cellular aggregate.

As used herein, “second nearest neighbor” volume partitions are the volume partitions surrounding the first nearest neighbor volume partitions.

As used herein, “third nearest neighbor” volume partitions are the volume partitions surrounding the second nearest neighbor volume partitions.

As used herein, an “oxidizing agent” is any molecule capable of oxidizing another molecule.

As used herein, a “reagent” is any molecule that is capable of undergoing a chemical reaction.

As used herein, a “reflectance characteristic” is the amount of light reflected by a sample. In some embodiments, a reflectance characteristic comprises frequency or wavelength of light, its polarization, and/or the angle of incidence.

As used herein, a “sample mixture” is a mixture comprising cells and growth media.

As used herein, “specificity” is the proportion of true negative results among the total number of true negative results and false positive results.

As used herein, “sensitivity” is the proportion of true positive results among the total number of true positive results and false negative results.

As used herein, a “spatial discretization mechanism” refers to any mechanism for partitioning individual samples that enables detection of concentration gradients as molecules diffuse between the individual samples. In some embodiments, the spatial discretization mechanism is an emulsified droplet containing the individual sample.

As used herein, a “static two-dimensional array” is an array of droplets generated using a differential that, once generated, is held still to continuously image over a period of time. In some embodiments, the differential is a pressure differential.

As used herein, “hemolytic behavior” is the breakdown of red blood cells by bacterial cells.

### Characterizing

The disclosure provides methods for characterizing cells.

In some embodiments, characterizing cells comprises classifying or identifying the cells.

In some embodiments, characterizing cells comprises identifying one or more phenotypic characteristics of the cells. Exemplary phenotypic characteristics comprise hemolytic behavior, gram stain, antibiotic susceptibility, and sugar metabolism. Further phenotypic characteristics are known in the art.

In some embodiments, the cells are classified by gram stain.

In some embodiments, the genus of the cells is identified.

In some embodiments, the species of the cells is identified.

In some embodiments, both the genus and species of the cells is identified.

In some embodiments, characterizing cells comprises identifying the presence or absence of a specific genus and/or species of the cell.

In some embodiments, characterizing cells comprises distinguishing between two or more cell types.

In some embodiments, characterizing cells comprises distinguishing between two or more cell types of the same genus.

In some embodiments, characterizing cells comprises clinically identifying cells.

In some embodiments, characterizing cells comprises determining the susceptibility of said cells to one or more molecules.

In some embodiments, the one or more molecules are antibiotics.

In some embodiments, characterizing cells comprises identifying a specific cell type in a monomicrobial context.

In some embodiments, characterizing cells comprises identifying one or more specific cell types in a polymicrobial context.

In some embodiments, characterizing cells comprises identifying two or more specific cell types in a polymicrobial context.

### Growth Media

In some embodiments, the methods of the disclosure comprise combining the cells with a growth media.

Exemplary growth media for different cell types are known in the art.

In some embodiments, the growth media is a bacterial cell growth media. A bacterial cell growth media may be any media that supports growth of one or more bacterial cells.

In some embodiments, the bacterial cell growth media comprises tryptic soy broth.

In some embodiments, the bacterial growth media supports growth of one of more Streptococci strains.

In some embodiments, the bacterial growth media supports growth of Streptococcus pneumoniae (S. pneumoniae).

In some embodiments, the bacterial growth media supports growth of Streptococcus mitis (S. mitis).

In some embodiments, the growth media is a eukaryotic cell growth media. A eukaryotic cell growth media may be any media that supports growth of one or more eukaryotic cells. Exemplary eukaryotic cell growth media are known in the art.

In some embodiments of the method disclosed herein, the growth media further comprises one or more reagents.

In some embodiments, the growth media comprises one reagent. In some embodiments, the growth media comprises two reagents. In some embodiments, the growth media comprises three or more reagents.

In some embodiments, one of the reagents is hemoglobin.

In some embodiments, one of the reagents is a sugar, a protein, a peptide, a nucleic acid, an active agent, a drug, a pharmaceutical drug, a cell (e.g., a prokaryotic cell or a eukaryotic cell), or one or more metabolites from a cell. Optionally where one of the reagents is a metabolite from a cell, it is from a different cell type to the cells being characterized.

Hydrogen peroxide undergoes a chemical reaction with hemoglobin to form the product oxidized hemoglobin. In some embodiments, the cells produce hydrogen peroxide, which is available to undergo a chemical reaction with hemoglobin when hemoglobin is a reagent component of the growth media.

In some embodiments, one or more of the reagents is a detection agent.

In some embodiments, the detection agent is a fluorescent dye.

In some embodiments, the fluorescent dye produces a fluorescent signal in its native state. In some embodiments, the fluorescent signal of the dye can be modified by a reaction. In some embodiments, the reaction is oxidation of the fluorescent dye. In some embodiments, the reaction decreases the fluorescent signal. In some embodiments, the reaction increases the fluorescent signal.

In some embodiments, the dye produces a fluorescent signal following a reaction. In some embodiments, said dye requires activation to produce a fluorescent signal.

In some embodiments, the fluorescent dye is a pyranine dye, a resazurin dye, a tetrazolium dye, a coumarin dye, an anthraquinone dye, a cyanine dye, an azo dye, a xanthene dye, an arylmethine dye, a pyrene derivative dye, a ruthenium bipyridyl complex dye, or a derivative thereof. As used herein, “derivative” is understood to mean a chemically modified form of a dye that maintains some of the detection characteristics, e.g., fluorescent sensitivity to redox state, of the compound from which it is derived. Further fluorescent dyes are known in the art.

In some embodiments, the fluorescent dye is pyranine.

In some embodiments, the detection agent is detectable by optical means (e.g., a fluorescent signal).

In some embodiments, the detection agent is detectable by non-optical means.

### Partitioning

In some embodiments, the methods of the disclosure comprise partitioning the sample mixture into a plurality of volume partitions, wherein some or each of the volume partitions contain(s) one cell or one cellular aggregate from the sample mixture.

In some embodiments, the volume partitions are droplets. Exemplary methods of forming droplets are set out in WO 2018/069866, which claims priority from U.S. Application No. 62/407,311, and is hereby incorporated by reference in its entirety.

In some embodiments, the volume partitions are droplets in an immiscible fluid. Upon mixing of the sample mixture and the immiscible fluid, they form phases—an aqueous drop or partition, which holds the sample mixture in solution, and a non-aqueous phase made up of the immiscible fluid. The immiscible fluid can be a fluorocarbon comprising a fluorosurfactant or hydrocarbon oils such as mineral oil, or silicone oils.

In some embodiments, the volume partitions are formed using Laplace pressure gradients (see, for example, Dangla et al., 2013, PNAS 110(3):853-58). An aqueous phase containing cells or microbes can be introduced into a device having a reservoir of a continuous phase (i.e., immiscible fluid) forming an aqueous “tongue” in an appropriate device. The device can incorporate height variation(s) into a microchannel that subject the immiscible interfaces to a difference in curvature between the portion of the aqueous phase that has not encountered the height variation and the portion of the aqueous phase downstream of the height variation. As the aqueous phase flows through the height variation, a critical curvature is reached for the portion of the aqueous phase downstream of the height variation beyond which the two portions cannot remain in static equilibrium, breaking off the aqueous phase into a droplet, as the downstream portion detaches from the tongue formed by introduction of the aqueous phase into a continuous phase, the size of the drops being determined by the device geometry. The height variation can be accomplished with a single step change in the height of a microchannel (step emulsification), multiple steps (multi-step emulsification), and a ramp or similarly gradual gradients of confinement.

In some embodiments, the volume partitions are formed using shear stress methods.

In some embodiments, the droplets are emulsified droplets.

In some embodiments, the volume partitions are formed from chambers separated by membranes. In some embodiments, the chambers are in a hexagonal arrangement.

In some embodiments, the number of droplets produced depends on the concentration according to Poisson statistics.

In some embodiments, the volume partitions have a volume of less than 1 μL.

In some embodiments, the volume partitions have a volume of less than 100 nL, 10 nL, 1 nL, 100 pL, 10 pL, 1 pL, 100 fL, 10 fL, or 1 fL.

In some embodiments, the volume partitions have a volume of 0.1 μL to 10 nL.

In some embodiments, the volume partitions have a volume of 25-500 pL.

In some embodiments, the volume partitions have a volume of 40-300 pL.

In some embodiments, the methods of the disclosure further comprise arranging the plurality of volume partitions on at least one array.

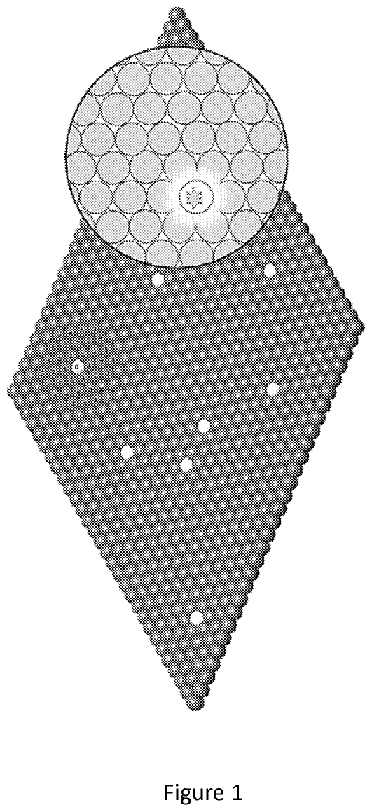

Methods of arranging volume partitions on an array are known in the art. FIG. 1 provides an exemplary array.

In some embodiments, the array is a two-dimensional array.

In some embodiments, the two-dimensional array is a static two-dimensional array.

In some embodiments, the array comprises two or more volume partitions.

In some embodiments, the array comprises ten or more, 100 or more, 1000 or more, 10000 or more, 15000 or more, 20000 or more, 25000 or more volume partitions.

In some embodiments, the cells are heterogeneously distributed among the volume partitions.

In some embodiments, one or more of the neighboring volume partitions does not comprise a cell or cellular aggregate.

### Incubation

In some embodiments, the methods of the disclosure comprise incubating the volume partitions for a time sufficient to permit (i) at least one initial reaction to occur and produce one or more products, and (ii) diffusion of one or more of the products into one or more neighboring volume partitions.

In some embodiments, the time is at least one minute.

In some embodiments, the time is at least 30 minutes.

In some embodiments, the time is at least one hour.

In some embodiments, the time is at least two hours.

In some embodiments, the time is at least four hours.

In some embodiments, the time is at least six hours.

In some embodiments, the time is at least eight hours.

In some embodiments, the time is at least 10 hours.

In some embodiments, the incubation time provides for rapid characterizing of cells compared to existing methods known in the art.

In some embodiments, the methods of the disclosure comprise incubating the volume partitions at one or more temperatures.

In some embodiments, the one or more temperatures allow cell proliferation.

In some embodiments, the one or more temperatures allow the at least one initial reaction to occur.

In some embodiments, the one or more temperatures allow a subsequent reaction to occur between one or more of the products and the detection agent(s).

In some embodiments, the volume partitions are incubated at one or more temperatures from 5° C. to 95° C. In some embodiments, the one or more temperatures are from 20° C. to 60° C.

In some embodiments, the one or more temperatures comprise 35° C. to 37° C. In some embodiments, the one or more temperatures comprise 35° C.

In some embodiments, the incubation time is sufficient to permit at least one initial reaction to occur to produce at least one product.

In some embodiments, the at least one initial reaction comprises a cellular metabolic pathway reaction and/or cellular respiration.

In some embodiments, the at least one initial reaction comprises a cellular metabolic pathway reaction.

In some embodiments, wherein the cells are prokaryotic cells, the cellular metabolic pathway reaction is a prokaryotic cellular metabolic pathway reaction.

In some embodiments, where the cells are Streptococcus pneumoniae, the cellular metabolic pathway reaction is Streptococcus pneumoniae metabolism.

In some embodiments the initial reaction produces hydrogen peroxide.

In some embodiments, where the cells are Streptococcus pneumoniae, hydrogen peroxide is produced by pyruvate oxidase and/or lactate oxidase.

In some embodiments, wherein the cells are eukaryotic cells, the cellular metabolic pathway reaction is a eukaryotic cellular metabolic pathway reaction.

In some embodiments, the at least one initial reaction comprises cellular respiration.

In some embodiments, the initial reaction produces at least one product.

In some embodiments, the at least one product is a product of metabolism. In some embodiments, the product of metabolism is a cell metabolite. Exemplary metabolites are known in the art.

In some embodiments, the cell metabolite is hydrogen peroxide.

In some embodiments, the at least one product is a product of cellular respiration. Exemplary products of cellular respiration are known in the art.

In some embodiments, the at least one product comprises an oxidizing agent.

In some embodiments, the oxidizing agent comprises oxidized hemoglobin.

In some embodiments, the at least one product is oxidized.

In some embodiments, the at least one or more of the products undergoes a further reaction.

In some embodiments, the further reaction comprises oxidation of one or more of the reagents.

In some embodiments, one or more of the products is reduced. In some embodiments, the reduced product acts as an oxidizing agent in the further reaction.

In some embodiments, the product (e.g., oxidized hemoglobin), is produced by a reaction between the product of the initial reaction (e.g., hydrogen peroxide) and one or more of the reagents (e.g., hemoglobin).

In some embodiments, one or more of the products of the initial reaction reacts with hemoglobin to form oxidized hemoglobin.

In some embodiments, the incubation time is sufficient to permit one or more of the products to diffuse into one or more neighboring volume partition(s).

In some embodiments, one or more of the products diffuses into at least one neighboring volume partition.

In some embodiments, one or more of the products diffuse into two or more neighboring volume partitions.

In some embodiments, neighboring volume partitions are in close proximity to the volume partition from which the one or more products diffuses.

In some embodiments, volume partitions are in close proximity if the volume partitions have a center-to-center distance of approximately the diameter of the volume partition or less.

In some embodiments, neighboring volume partitions are hexagonally closely-packed first, second, and third nearest neighbor volume partitions.

In some embodiments, neighboring volume partitions are hexagonally closely-packed first and second nearest neighbor volume partitions.

In some embodiments, neighboring volume partitions are hexagonally closely-packed first nearest neighbor volume partitions.

In some embodiments, neighboring volume partitions are immediately next to the volume partition from which the one or more products diffuses.

In some embodiments, neighboring volume partitions are within 100 μm of the volume partition from which the one or more products diffuses.

In some embodiments, neighboring volume partitions are within 90 μm, 80 μm, 70 μm, 60 μm, 50 μm, 10 μm, or 1 μm of the volume partition from which the one or more products diffuses.

In some embodiments, the detection agent(s) are modified in the volume partition, and modified detection agent(s) diffuse to one or more neighboring volume partitions.

In some embodiments, the modified detection agent(s) produce a modified, increased, or decreased fluorescent signal.

### Monitoring and Characterizing

In some embodiments, the methods of the disclosure comprise monitoring the neighboring volume partitions for one or more characteristics that results from a subsequent reaction between one or more of the products and the detection agent(s).

In some embodiments, the methods of the disclosure provide methods to detect differential production of metabolites by cells.

In some embodiments, the methods of the disclosure detect differential production of metabolites through the diffusion of molecules and/or macromolecules between volume partitions.

In some embodiments, diffusion occurs via a spatial discretization mechanism. In some embodiments, the spatial discretization mechanism comprises movement between two or more compartments wherein there is a gradient between the two or more compartments. In some embodiments, the mechanism retains communication between the two or more compartments and produces a diffusion gradient. In some embodiments, the mechanism reduces the speed of diffusion.

In some embodiments, the spatial discretization mechanism comprises droplets with oil and/or surfactant between the droplets.

In some embodiments, the spatial discretization mechanism comprises a solid matrix. In some embodiments, the solid matrix comprises an agar plate.

In some embodiments, monitoring at least one characteristic comprises monitoring an amount of signal produced by a reaction between one or more of the products and the detection agent(s).

In some embodiments, monitoring comprises measuring and/or recording a change in the amount of signal from a detection agent.

In some embodiments, monitoring comprises measuring and/or recording an amount of signal produced by a reaction.

In some embodiments, signal is measured using a bandpass-filtered, Stokes-shifted light with a camera. Alternative methods of measuring and recording a signal are known in the art.

In some embodiments, the reaction is between one or more of the products and the detection agent(s).

In some embodiments, one or more of the products reacts with one or more of the reagents to produce a modified product or modified reagent that reacts with the detection agent(s).

In some embodiments, the reaction of one or more of the products with one or more of the reagents occurs in one or more of the neighboring volume partitions.

In some embodiments, the one or more products oxidize one or more of the reagents, to produce one or more oxidized reagents. The one or more oxidized reagents then oxidize the detection agent(s). In some embodiments, oxidation of the detection agent(s) modifies the fluorescent signal of the detection agent(s).

In some embodiments, the product is hydrogen peroxide, and the reagent is hemoglobin. In some embodiments, hydrogen peroxide oxidizes hemoglobin to produce oxidized hemoglobin. In some embodiments, oxidized hemoglobin oxidizes the detection agent(s).

In some embodiments, the reaction is between an oxidizing agent and the detection agent(s). In some embodiments, the oxidizing agent oxidizes the detection agent(s) to produce one or more oxidized detection agents.

In some embodiments, the reaction is between oxidized hemoglobin and the detection agent(s).

In some embodiments, wherein the detection agent is a fluorescent dye, the reaction between the oxidizing agent (e.g., oxidized hemoglobin) and the dye (e.g., pyranine), produces an oxidized dye.

In some embodiments, the reaction between the at least one product and the detection agent(s) modifies a characteristic of the detection agent(s).

In some embodiments, the detection agent is modified by oxidation.

In some embodiments, modifying one or more characteristics comprises decreasing a characteristic of the detection agent(s).

In alternative embodiments, modifying one or more characteristics comprises increasing, decreasing, and/or modifying a characteristic of the detection agent(s).

In some embodiments, monitoring one or more characteristics comprises constructing a waveform for each volume partition, wherein the waveform reflects the change of the characteristic over time.

In some embodiments, the waveform for each volume partition is constructed from signals measured and/or recorded at two or more time points. In some embodiments, the waveform is constructed from three or more, five or more, 10 or more, 50 or more, 100 or more, 150 or more, 200 or more, 500 or more, or 1000 or more signals.

In some embodiments, different cell types produce products that interact with reagents to produce different waveforms from the detection agents.

In some embodiments, different cell types produce products (e.g., cell metabolites) that interact with a reagent (e.g., hemoglobin), on different timescales, to generate different waveforms for characterizing cells.

In some embodiments, the characteristics comprise optical characteristics and/or chemical characteristics. Exemplary optical and chemical characteristics are known in the art.

In some embodiments, the optical characteristics comprise fluorometric, colorimetric, and/or reflectance characteristics.

In some embodiments, the detection agent is a fluorescent dye and the fluorometric characteristic comprises fluorescent signal of the dye.

In some embodiments, the waveform is constructed from fluorescent signals.

In some embodiments, monitoring one or more optical characteristics comprises monitoring for a decrease in one or more optical characteristics in the neighboring partitions. In some embodiments, monitoring one or more optical characteristics comprises monitoring for a decrease in fluorescent signal.

In some embodiments, the reaction between (i) one or more of the products and/or (ii) one or more of the reagents, and the fluorescent dye decreases the fluorescent signal of the fluorescent dye.

In some embodiments, modifying one or more of characteristics comprises monitoring a modification (e.g., an increase or a decrease) compared to a measurement taken in a control sample mixture that is known to not comprise cells.

In some embodiments, the first reading of the waveform is the control reading against which modification is monitored.

In some embodiments, the control sample comprises growth media.

In some embodiments, modifying one or more of the characteristics

comprises a modification (e.g., an increase or a decrease) to a measurement taken in the volume partition before incubating the volume partitions.

Methods of modifying characteristics (e.g., optical characteristics and/or non-optical characteristics) are known in the art.

In some embodiments, the methods of the disclosure comprise characterizing the cells in the sample according to the methods of the disclosure.

In some embodiments, characterizing the cells in the methods of the disclosure is provided through monitoring the neighboring volume partitions for one or more characteristics that results from a reaction between (i) one or more of the products and/or (ii) one or more of the reagents, and the detection agent(s).

In some embodiments, the detection range is from 10³ cells/mL to 10¹² cells/mL. In some embodiments, the detection range is from 10⁴ to 10¹¹ cells/mL, from 10⁴ to 10¹⁰ cells/mL, from 10⁴ to 10⁹ cells/mL, from 10⁴ to 10⁸ cells/mL, or from 10⁴ to 10⁷ cells/mL. In some embodiments, the detection range is from 10⁴ to 10⁶ cells/mL. In some embodiments, the detection range for the method is from about 10⁴ cells/mL to about 7×10⁵ cells/mL.

### Cells

The methods of the disclosure are directed to characterizing cells.

In some embodiments, the cells are prokaryotic cells and/or eukaryotic cells.

In some embodiments, the prokaryotic cells are bacterial cells.

In some embodiments, the bacterial cells are cells of a genus and/or species associated with infection of a subject, optionally a human and/or animal subject.

In some embodiments, the bacterial cells comprise Streptococci. In some embodiments, the Streptococci are Streptococcus pneumoniae, Streptococcus mitis, Streptococcus pyogenes, Streptococcus agalactiae, Streptococcus dysgalactiae, Streptococcus anginosus, Streptococcus constellatus, Streptococcus salivarius, Streptococcus oralis, and/or other species typically found in normal oral flora).

In some embodiments, the Streptococci are Streptococcus pneumoniae and/or Streptococcus mitis.

In some embodiments, the disclosure is directed to a method for determining the presence of Streptococcus pneumoniae cells in a sample.

In some embodiments, said method comprises combining the sample with a growth media comprising hemoglobin, and a fluorescent dye, thereby forming a sample mixture. In some embodiments, the growth media comprises tryptic soy broth. In some embodiments, the fluorescent dye comprises pyranine.

In some embodiments, said method further comprises partitioning the sample mixture into a plurality of membrane emulsified droplets, wherein some or each of said droplets contains one cell or one cellular aggregate from the sample mixture.

In some embodiments, said method further comprises arranging the plurality of droplets on at least one array.

In some embodiments, said method further comprises incubating the droplets for a time sufficient to permit (i) metabolism of the Streptococcus pneumoniae cells to produce hydrogen peroxide, (ii) a reaction between the hydrogen peroxide and the hemoglobin to produce oxidized hemoglobin, (iii) a reaction between the oxidized hemoglobin and the fluorescent dye to produce an oxidized dye, and (iv) diffusion of at least one of the hydrogen peroxide, the oxidized hemoglobin, or the oxidized dye into one or more neighboring droplets.

In some embodiments, said method further comprises monitoring a fluorescent signal of the one or more neighboring droplets during the incubation.

In some embodiments, said method further comprises determining the presence or absence of Streptococcus pneumoniae cells in the sample.

In some embodiments, the eukaryotic cells are yeast cells. Exemplary yeast cells are Candida (e.g., C. albicans). In some embodiments, the yeast cells are of the Cryptococcus genus, e.g., C. neoformans. Clinically relevant eukaryotic cells are known in the art.

In some embodiments, the cells comprise monomicrobial cells.

In some embodiments the cells are obtained from blood. In some embodiments, the blood is a blood sample. Methods of obtaining blood samples are known in the art.

In some embodiments, the cells are polymicrobial cells. In some embodiments, the polymicrobial cells comprise two or more cells of different cell types.

In some embodiments, the polymicrobial cells comprise two different cell types of the same genus.

In some embodiments, the two different cell types are of the genus Streptococcus. In some embodiments, the cells comprise S. pneumoniae cells and S. mitis cells.

In some embodiments, the cells are obtained from a respiratory sample. Methods of obtaining respiratory samples are known in the art.

### Sensitivity and Specificity

In some embodiments, the methods of the disclosure provide higher sensitivity than existing methods.

In some embodiments, sensitivity is calculated as a percentage.

In some embodiments, the sensitivity of the methods of the disclosure is at least 90%. In some embodiments, the sensitivity is at least 93%, at least 95%, at least 96%, at least 97%, at least 98%, at least 99%, or 100%.

In some embodiments, the sensitivity of the methods of the disclosure is 93-100%.

In some embodiments, the methods of the disclosure provide higher specificity than existing methods.

In some embodiments, specificity is calculated as a percentage.

In some embodiments, the specificity is at least 85%. In some embodiments, the sensitivity is at least 90%, at least 91%, at least 92%, at least 93%, at least 94%, at least 95%, at least 96%, at least 97%, at least 98%, at least 99%, or 100%.

In some embodiments, the methods of the disclosure provide higher sensitivity and higher specificity than existing methods.

In some embodiments, the sensitivity is at least 93%, and the specificity is at least 85%.

In some embodiments, the sensitivity is at least 93%, and the specificity is 100%.

### EXAMPLES

The disclosure is further illustrated by the following non-limiting examples. The examples serve only for illustration, and are not intended to limit the scope of the disclosure, which is as set out in the claims.

### Example 1

Example 1 provides an exemplary method according to the disclosure for characterizing S. pneumoniae cells. Example 1 further provides data demonstrating that the methods of the disclosure provide a rapid, sensitive, and selective method for characterizing cells.

Streptococcus pneumoniae are very difficult to differentiate from other alpha-hemolytic Streptococcus species (e.g., Streptococcus mitis) using existing methods, due to high sequence similarity. Streptococcus pneumoniae and other alpha-hemolytic Streptococcus species (e.g., Streptococcus mitis) share more than 99% sequence homology.

Therefore, characterizing cells as Streptococcus pneumoniae is difficult in the monomicrobial context.

Characterizing cells as Streptococcus pneumoniae is even more difficult in the polymicrobial context. It is common for samples, such as clinical samples obtained from subjects with a bacterial infection, to comprise both Streptococcus pneumoniae and Streptococcus mitis.

According to the methods of the disclosure, cells were combined with a tryptic soy broth at a concentration of about 10⁵ cells/mL to form a sample mixture. Tryptic soy broth is a bacterial cell growth media comprising all components required for Streptococcus pneumoniae cell growth. The sample mixture further comprised about 5 μM of the reagent hemoglobin, and about 80 UM of the detection agent pyranine.

The sample mixture was partitioned into a plurality of emulsified droplets and arranged on an array. The 10⁵ cells/mL produced about 400 occupied droplets in the array, each containing a single cell or a single cellular aggregate.

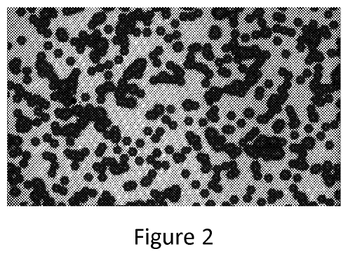

Following incubation at 37° C. for 6 hours, a decrease in fluorescent signal was observed in droplets in which Streptococcus pneumoniae cells were encapsulated, and neighboring droplets (FIG. 2).

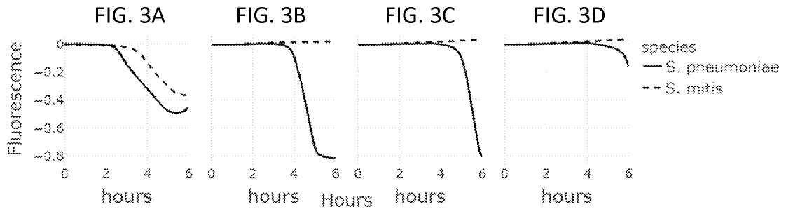

Waveforms were generated for the emulsified droplets from fluorescent signal readings taken every 2.5 minutes for 6 hours. FIG. 3 depicts the waveforms of emulsified droplets comprising Streptococcus pneumoniae or Streptococcus mitis. FIG. 3 depicts differential signals from a representative strain of Streptococcus pneumoniae and a representative strain of Streptococcus mitis.

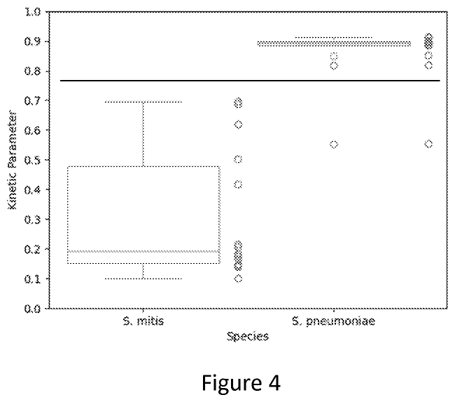

FIG. 4 demonstrates that the waveforms exemplified in FIG. 3 can be used to distinguish between different species and to characterize cells. FIG. 4 demonstrates that the kinetic parameter depicted effectively distinguishes between Streptococcus pneumoniae cells and Streptococcus mitis cells.

The solid line between the kinetic parameter values 0.7 and 0.8 provides an exemplary means by which an algorithm could distinguish between different species and characterize cells.

The method exemplified in FIG. 4 of claim 4 achieves 93-100% sensitivity, as depicted in Table 1 of Example 2.

The single datapoint below the solid line for Streptococcus pneumoniae results from inconclusive bile solubility identification testing for Streptococcus pneumoniae. Including this datapoint in the analysis results in 93% sensitivity of the exemplified method. Excluding this datapoint from the analysis results in 100% sensitivity of the exemplified method.

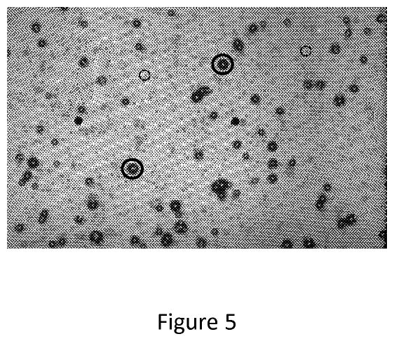

FIG. 5 demonstrates that the fluorescent signal decreased about 10× more in neighboring emulsified droplets for droplets containing Streptococcus pneumoniae cells compared to droplets containing Streptococcus mitis cells in the same sample. Droplets containing Streptococcus mitis cells were identified in a separate optical channel and are indicated in FIG. 5 by dashed circles. Droplets containing Streptococcus pneumoniae are indicated in FIG. 5 by solid circles.

Example 1 demonstrates that neighboring encapsulated droplets produced different waveforms for droplets comprising Streptococcus pneumoniae compared to droplets comprising Streptococcus mitis.

Therefore, Example 1 demonstrates that the methods of the disclosure provide methods for characterizing cells with high sensitivity and specificity.

### Example 2

Example 2 demonstrates the methods of the disclosure provide an improvement compared to existing methods for characterizing cells.

The exemplary method of the disclosure described in Example 1 was compared to existing methods for characterizing cells, and demonstrated that the methods of the disclosure provide improved sensitivity and specificity compared to existing methods.

The method of Example 1 was compared to MALDI-TOF MS (Yahiaoui et al., 2020, Clinical Microbiology and Infection 26(8)), bile solubility (Richter et al., 2008, Journal of Clinical Microbiology 46(7):1088.e1-1088.e5), optochin (Richter et al., 2008, Journal of Clinical Microbiology 46(7):1088.e1-1088.e5), and Verigene (Wojewoda et al., 2013, Journal of Clinical Microbiology 51(7):2-72-6) methods for characterizing cells.

The methods of the disclosure provide rapid characterization of cells compared to known methods.

The methods of the disclosure provide characterization of cells in polymicrobial samples.

The methods of the disclosure provide characterization of cell types that cannot be characterized using existing methods.

Table 1 sets out the comparative percentage sensitivity and percentage specificity data for the method of Example 1 and the existing methods.

TABLE 1

| | Sensi- | Speci- | |
|---|---|---|---|
| | tivity | ficity | |
| Method | (%) | (%) | Strains |
| Method of | 93-100 | 100 | 13 Streptococcus pneumoniae |
| the disclosure | | | isolates 35 viridans group |
| according to | | | Streptococci |
| Example 1 | | | |
| MALDI-TOF | 99.8 | 44.6 | 496 Streptococcus pneumoniae |
| MS | | | isolates 83 viridans group |
| | | | Streptococci |
| Bile Solubility | 98.8 | 82.6 | 1647 Streptococcus pneumoniae |
| | | | isolates 86 viridans group |
| | | | Streptococci |
| Optochin | 87.9 | 59.3 | 1647 Streptococcus pneumoniae |
| | | | isolates 86 viridans group |
| | | | Streptococci |
| Verigene | 100 | 40 | 2 Streptococcus pneumoniae |
| | | | positive blood cultures |
| | | | 3 Streptococcus mitis positive |
| | | | blood cultures |

The comparative data set out in Table 1 demonstrate that the methods of the disclosure provide improved sensitivity and specificity compared to existing methods.

As depicted in Table 1, some of the existing methods may provide high sensitivity or high specificity. However, none of the existing methods provide both high sensitivity and high specificity.

Example 2 demonstrates that the methods of the disclosure provide high sensitivity and high specificity, and represent an improvement over existing methods.

Example 2 also demonstrates that the methods of the disclosure provide a rapid method for characterizing cells compared to existing methods.

Example 2 also demonstrates that the methods of the disclosure provide methods of characterizing cells in polymicrobial samples.

Example 2 also demonstrates that the methods of the disclosure characterize S. pneumoniae cells.

### Example 3

Example 3 demonstrates an exemplary method of the disclosure.

The detection agent is a dye that changes color to characterize cells, such as methylene blue.

The cells are combined with a growth media comprising one or more reagents and the dye to form a sample mixture.

The sample mixture is partitioned into a plurality of volume partitions, wherein some of each of the volume partitions contain one cell or one cellular aggregate from the sample mixture, and incubated. The volume partitions are droplets as described in Example 1, and the volume partitions are incubated as described in Example 1.

The cells react with one or more of the reagents and change the reduction state of the dye. Upon oxidation or reduction, the dye changes color, and the cells are characterized by detection of the changes in absorbance of the dye at a particular wavelength, which for methylene blue is 660 nm.

The color change is measured using a spectrophotometer.

The color change of the dye allows the characterization of cells.

For example, the change in absorbance for S. pneumoniae may differ to the change in absorbance for S. mitis.

This example demonstrates characterization of cells by a colorimetric method according to the disclosure.

### Example 4

The method of the disclosure provides a method of characterizing mammalian cells by characterizing oxidative stress.

The mammalian cells are combined with a growth media comprising one or more reagents, wherein one or more of the reagents is a detection agent to form a sample mixture.

The exemplary mammalian growth media comprises Dulbecco's Modified Eagle Medium (DMEM).

The sample mixture is partitioned into a plurality of volume partitions, wherein some or each of the volume partitions contain one cell or one cellular aggregate from the sample mixture, and incubated. The volume partitions are droplets as described in Example 1, and the droplets are incubated.

Modification of the detection agent provides differential reactions to oxidative damage, and the method characterizes cells based on oxidative damage.

The oxidative damage is induced by a drug, a pathogen, or other stressor, wherein the method of the disclosure characterizes cells based on their response to said drug, pathogen or other stressor and/or the presence of said drug, pathogen or other stressor.

The method of Example 4 characterizes mammalian cells based on their response to a stressor, such as a drug or pathogen.

### EQUIVALENTS

Those skilled in the art will recognize many equivalents to the specific embodiments of the disclosure escribed herein. The scope of the present disclosure is not intended to be limited to the above Description, but rather is as set forth in the claims.

1. A method for characterizing cells comprising:
(a) combining the cells with a growth media comprising one or more reagents, thereby forming a sample mixture, wherein one or more of the reagents is a detection agent;
(b) partitioning the sample mixture into a plurality of volume partitions, some or each of said volume partitions containing one cell or one cellular aggregate from the sample mixture;
(c) arranging the plurality of volume partitions on at least one array;
(d) incubating the volume partitions for a time sufficient to permit (i) at least one initial reaction to occur and produce one or more products, and (ii) diffusion of one or more of the products into one or more neighboring volume partition(s),
(e) monitoring the neighboring volume partitions for one or more characteristics that results from a subsequent reaction between one or more of the products and the detection agent(s); and
(f) thereby characterizing the cells in the sample.

2. The method of claim 1, wherein the volume partitions are emulsified droplets.

3. The method of claim 1, wherein incubating the volume partitions comprises incubating at one or more temperatures.

4. The method of claim 1, wherein one or more of the products is a cell metabolite and/or one or more of the products is an oxidizing agent.

5. The method of claim 4, wherein the metabolite is hydrogen peroxide and/or the oxidizing agent is oxidized hemoglobin.

6. The method of claim 5, wherein one of the reagents is hemoglobin.

7. The method of claim 1, wherein monitoring the one or more characteristics comprises monitoring an amount of signal produced by a reaction between one or more of the products and the detection agent(s).

8. The method of claim 1, wherein the volume partitions have a volume of less than 1 μL.

9. The method of claim 8, wherein the volume is less than 100 nL, 10 nL, 1 nL, 100 pL, 10 pL, 1 pL, 100 fL, 10 fL, or 1 fL.

10. The method of claim 1, wherein one or more of the characteristics are optical characteristics and/or chemical characteristics.

11. The method of claim 10, wherein the optical characteristics are fluorometric, colorimetric, and/or reflectance characteristics.

12. The method of claim 11, wherein monitoring the optical characteristics comprises monitoring for a decrease in fluorescent signal in the neighboring volume partitions.

13. The method of claim 1, wherein monitoring one or more of the characteristics comprises constructing a waveform for each volume partition, wherein such a waveform reflects the change of one or more of the characteristics over time.

14. The method of claim 1, wherein the detection agent is a fluorescent dye.

15. The method of claim 14, wherein the fluorescent dye is pyranine.

16. The method of claim 1, wherein the cells are heterogeneously distributed among the volume partitions.

17. The method of claim 1, wherein the cells are bacterial cells.

18. The method of claim 17, wherein the bacterial cells are Streptococcus pneumoniae.

19. The method of claim 1, wherein the growth media is tryptic soy broth.

20. The method of claim 1, wherein the time sufficient to permit the at least one reaction and the diffusion to occur is at least 2 hours, at least 4 hours, or at least 6 hours.

21. A method for determining the presence of Streptococcus pneumoniae cells in a sample comprising:
(a) combining the sample with a growth media comprising tryptic soy broth, hemoglobin and a fluorescent dye forming a sample mixture;
(b) partitioning the sample mixture into a plurality of membrane emulsified droplets, some or each of said droplets containing one cell or one cellular aggregate from the sample mixture;
(c) arranging the plurality of droplets on at least one array;
(d) incubating the droplets for a time sufficient to permit (i) metabolism of the Streptococcus pneumoniae cells to produce hydrogen peroxide, (ii) a reaction between the hydrogen peroxide and the hemoglobin to produce oxidized hemoglobin, (iii) a reaction between the oxidized hemoglobin and the fluorescent dye to produce an oxidized dye, and (iv) diffusion of at least one of the hydrogen peroxide, the oxidized hemoglobin, or the oxidized dye into one or more neighboring droplets,
(e) monitoring a fluorescent signal of the one or more neighboring droplets during the incubation; and
(f) thereby determining the presence or absence of Streptococcus pneumoniae cells in the sample.

22. The method of claim 21, wherein the fluorescent dye comprises pyranine.
